(19) United States
(12) Patent Application Publication
(10) Pub. No.: US 2008/0015241 A1
(43) Pub. Date: Jan. 17, 2008

(54) All day rhinitic condition treatment regimen

(75) Inventor: Donna F. White, Louisville, KY (US)

Correspondence Address: INTELLECTUAL PROPERTY / TECHNOLOGY LAW, PO BOX 14329, RESEARCH TRIANGLE PARK, NC, 27709, US

(73) Assignee: Cornerstone BioPharma, Inc.

(21) Appl. No.: 11/485,931

(22) Filed: Jul. 13, 2006

(51) Int. Cl. A61K 31/4172; A61K 31/137

(52) U.S. Cl. 514/400; 514/649

(57) ABSTRACT

A therapeutic regimen and method of treating a rhinitic condition is provided comprising a first pharmaceutical dosage form comprising a first therapeutically effective amount of an antihistamine and a therapeutically effective amount of an anticholinergic; a second pharmaceutical dosage form comprising a second therapeutically effective amount of an antihistamine and a therapeutically effective amount of the anticholinergic, where the second antihistamine has a greater H₁ receptor activity or a greater sedative effect than the first antihistamine. The regimen and method comprise indicia for distinguishing between the first and second pharmaceutical dosage forms, wherein the indicia distinguishes the first pharmaceutical dosage form for daytime administration and the second pharmaceutical dosage form for nighttime administration, and a prepackaged dispenser prefilled with the pharmaceutical dosage forms and comprising the indicia.

### FIELD OF THE INVENTION

The present invention relates to rhinitis treatment regimens, more particularly, to prepackaged therapeutic regimens for the symptomatic treatment of rhinitic conditions which combine antihistamine and anticholinergic active agents in a manner so as to avoid undesired augmentation of hypertension in a subject.

### BACKGROUND

Two types of oral medication are commonly used to treat rhinitis: decongestants and antihistamines. Decongestants and antihistamines differ in mechanism of action, therapeutic effects, and side effects. Many commercial rhinitis remedies combine the use of decongestants and antihistamines to bring about more complete symptom relief of rhinitis than with either medicament alone. Failure to treat symptoms of rhinitis may lead to other disorders including infection of the sinuses, ears and lower respiratory tract.

Decongestants are available both orally and topically as either short or long-acting preparations. Often combined with antihistamines, decongestants may help offset the soporitic effect of antihistamines. Decongestants may be tolerated during the day, and even be considered desirable to counter soporific effects, which are known to accompany other symptoms of rhinitis. Decongestants, however, may produce nervousness, restlessness, and insomnia if taken at night. This may confuse individuals to mistakenly attribute their inability to sleep to the malaise accompanying rhinitis rather than to the decongestant medication. Moreover, decongestants, for example, phenylephrine, pseudoephedrine and phenylpropanolamine, produce a narrowing of blood vessels which on one hand may lead to clearing of nasal congestion, but on the other hand, may cause an increase in blood pressure in patients who have high blood pressure.

Decongestants commonly used to treat rhinitis are predominately sympathomimetic agents. Sympathomimetic agents are generally a class of drugs whose pharmacology may include mimicking a stimulated sympathetic central nervous system. The effect of sympathomimetic agents may include increased cardiac output, dilation of bronchioles, and constriction of blood vessels. Sympathomimetic decongestants include, for example phenylephrine, pseudoephedrine and phenylpropanolamine. These agents act to constrict vessels in the nasal mucus membranes and thereby decrease tissue swelling and nasal congestion. In recent years certain sympathomimetic decongestants, for example, phenylpropanolamine, have been removed from OTC formulations because of implicated causation with hemorrhagic stroke.

Decongestants may be better than antihistamines for restoring the patency of congested nasal airways. However, like adrenaline, sympathomimetic nasal decongestants are stimulatory and generally may produce undesirable side effects. These undesirable side effects may include high blood pressure, heart irregularity and a general inability to tolerate decongestants.

Histamine is a mediator released from cells which line the walls of the nasal mucous membranes (mast cells). When released, histamine is known to bind to local receptors and thereby cause sneezing, nasal itching, swelling of the nasal membranes, and increased nasal secretions. Antihistamines relieve these effects, albeit by a different mechanism than decongestants. Antihistamines block the binding of histamine to histamine receptors in the nasal membranes, and thus function as H₁ receptor antagonists or inverse agonists. Antihistamines preemptively bind to histamine receptors and are effective only if given prior to histamine release because once histamine is released and binds to the receptor, administration of an antihistamine is generally ineffective. Although individuals may typically take antihistamines after symptoms occur, it is more desirable to dose antihistamine so as to effect therapeutic activity in anticipation of the peak times of histamine release. Individuals with rhinitic conditions commonly experience peak symptoms in the morning hours on awakening, a time believed to be concomitant with peak histamine release and coinciding with peak exposure to airborne allergens which would generally stimulate histamine release in sensitive individuals.

Anticholinergics generally block impulses from the part of the nervous system that controls heartbeat, blood pressure, and other responses to stress, by neutralizing the effects of acetylcholine. Anticholinergics include, for example, atropine, hyoscyamine, glycopyrrolate, methscopolamine, and scopolamine. Anticholinergics may help produce a drying effect in the nose and chest. Methscopolamine nitrate is a quaternary ammonium derivative of scopolamine, which possesses the peripheral actions of the belladonna alkaloids, but lacks the ability to cross the blood brain barrier, which is desirable. Methscopolamine nitrate may be used in combination with antihistamine because of its antisecretory effects on the respiratory system.

The combining of decongestants and antihistamines are generally known. The combination of decongestants and antihistamines may utilize a mechanistic approach where, for example, the incorporation of sympathomimetic decongestant and sedating antihistamine into a single dosage attempts to balance sympathomimetic stimulation and sedation of the respective components to possibly provide more complete relief of rhinitic symptomology than therapy with either component alone. Consequently, many products have been formulated so that their pharmaceutical dosage forms contain both a sympathomimetic decongestant and a sedating antihistamine.

While individuals are known to vary in their susceptibility to side effects, the incorporation of sympathomimetic decongestants and sedating antihistamine into a single dosage may cause some individuals to experience increased hypertension, irritability and/or sedation with these combinations. Examples of commercial formulations containing sympathomimetic decongestant and a sedating antihistamine include for example, BROMFED®, BENADRYL® and TAVIST-D®.

Formulations which incorporate both a sympathomimetic decongestant and a non-sedating antihistamine into a single dosage are generally known. While such formulations offer the advantage in being non-sedating, such combinations might be expected to provoke a greater incidence of risk to subjects with hypertension and may also exacerbate nighttime irritability and insomnia because the stimulating side effects of the decongestant may not be attenuated by concomitant use of sedating antihistamine. By way of example, the non-sedating antihistamine terfenadine, an active ingredient of SELDANE®, was removed from the market. A 25% incidence of insomnia has been reported among users of the combination of the non-sedating antihistamine terfenadine and the sympathomimetic pseudoephedrine decongestant. Examples of sympathomimetic decongestant and a non-sedating antihistamine formulations include, for example, CLARITIN-D®, CLARINEX-D® and ALLEGRA-D™.

Prepackaged regimens for treating the symptoms of rhinitis have been disclosed which employ a sympathomimetic decongestant for daytime and a decongestant plus a sedating antihistamine for night. See for example, U.S. Pat. No. 4,295,567, issued to Knudsen. Commercially available examples of the just mentioned regimens include: ACTIFED® and CONTAC DAY & NIGHT ALLERGY/SINUS®, both of which contains a sympathomimetic decongestant and a sedative antihistamine.

Prepackaged regimens have also been disclosed which incorporate a decongestant for daytime but not for nighttime, for example, SYN-RX™, which contains a sympathomimetic decongestant in the day formulation, and a non-sympathomimetic decongestant in the nighttime formulation. These regimens avoid stimulation from decongestant at night, however, they lack an antihistamine. Further, they neither contain medication which would be effective for rhinitis symptoms at night, nor anticipate peak rhinitic symptoms in the morning hours on awakening or formulate antihistamine pharmacological profiles to maximize therapeutic effectiveness while minimizing sedative side effects.

The short duration of sedation in relation to the longer duration of symptom suppression would suggest dosing of a sedative antihistamine at bedtime as a way to anticipate peak morning histamine release, and effectively confer combined antihistamine and decongestant activity during the day without sedation and without dosing of sedating antihistamine during the day. The limiting of stimulating decongestants during the day may avoid the potential for stimulation and insomnia at night. Such dosage regimens are disclosed in U.S. Pat. No. 6,270,796, U.S. Pat. No. 6,651,816 and U.S. Pat. No. 6,843,372 to Weinstein. Weinstein, however, requires in the disclosed regimen a sympathomimetic decongestant in at least one of the dosages and thus fails to address the risk associated with administering these decongestants to individuals with hypertension.

Thus, while the problem of sedation with combined decongestant and sedating antihistamine treatment and the problem of nighttime irritability and insomnia have been addressed, avoiding hypertension while effecting rhintic relief has not. Indeed, all sympathomimetic decongestant and antihistamine regimens have the potential to cause hypertension in addition to irritability and insomnia at night.

Similarly, the problem of sedation with combined sympathomimetic decongestant and sedating antihistamine treatment might be addressed in single entity combinations which employ sympathomimetic decongestants and non-sedating antihistamines, however, the problem of hypertension would not. Formulations which incorporate sympathomimetic decongestant and non-sedating antihistamine into a single pharmaceutical dosage form may be more likely to produce hypertension in addition to irritability and insomnia at night than formulations without sympathomimetic decongestants.

It is well known that individuals with rhinitis utilize antihistamines and decongestants hundreds of millions of times a year. It is not uncommon for inappropriate choices to result in symptomatic worsening rather than improvement. Individuals often use sedating medication unknowingly or inappropriately. Sympathomimetic decongestants taken at night not only may produce insomnia in a sizable portion of users, but may also cause daytime irritability, fatigue, and malaise. Users may mistakenly ascribe such symptoms to rhinitis rather than to medication. Professional as well as consumer confusion is widely encountered with these medications and unnecessarily negative consequences occur both by self-selection and prescription. There is a present need for preformulated regimens which advantageously use antihistamines and anticholinergics without sympathomimetic decongestants for rhinitic conditions in a manner to circumvent this confusion, avoid hypertension as well as reduce undesirable daytime sedation and/or eliminate nighttime stimulation.

Adherence to medication therapy and prevention of medication error are considerable medical problems and may be improved by establishing simplicity, reducing confusion, and increasing convenience. The proposed use of a multiplicity of pharmaceutical dosage forms as a regimen may be associated with pharmaceutical dosage forms being confused with each other, inadvertently switched, lost, misplaced, or ignored. Another problem associated with treatment using a plurality of pharmaceutical dosage forms is the lack of indicia which distinguish, signify and verify the pharmaceutical dosage forms from each other and their use together.

With more and more Americans becoming hypertensive, the need for new or improved rhinitis treatment regimens is becoming more of an issue. As many people cannot take decongestants because of concomitant disease states such as high blood pressure, heart irregularity and inability to tolerate decongestants, the present invention addresses this concern via a combination of active agents and treatments for rhinitic conditions with reduced or eliminated adverse hypertensive side effects.

### SUMMARY

In one embodiment, a therapeutic regimen is provided. The therapeutic regimen comprises a first pharmaceutical dosage form comprising a therapeutically effective amount of a first antihistamine having H₁ receptor activity and a therapeutically effective amount of an anticholinergic, and a second pharmaceutical dosage form comprising a therapeutically effective amount of a second antihistamine having H₁ receptor activity and a therapeutically effective amount of the anticholinergic with the proviso that the second antihistamine is not acrivastine, astemizole, cetirizine, loratadine, mizolastine or fexofenadine. The second antihistamine H₁ receptor activity is greater than the first antihistamine H₁ receptor activity. The therapeutic regimen comprises indicia for distinguishing between the first and second pharmaceutical dosage forms, where the indicia distinguishes the first pharmaceutical dosage form for daytime administration and the second pharmaceutical dosage form for nighttime administration. A prepackaged dispenser prefilled with the pharmaceutical dosage forms and comprising the indicia is also provided.

In another embodiment, a method is provided for treating the symptoms of a rhinitic condition. The method comprises the steps of formulating a rhinitic treatment regimen comprising a first pharmaceutical dosage form comprising a therapeutically effective amount of a first antihistamine having H₁ receptor activity and a therapeutically effective amount of an anticholinergic, and a second pharmaceutical dosage form comprising a therapeutically effective amount of a second antihistamine having H₁ receptor activity and a therapeutically effective amount of the anticholinergic. The second antihistamine H₁ receptor activity is greater than the first antihistamine H₁ receptor activity. The method provides indicia for distinguishing between the first and second pharmaceutical dosage forms, where the indicia distinguishes the first pharmaceutical dosage form for daytime administration and the second pharmaceutical dosage form for nighttime administration and prefilling a pharmaceutical dispenser with the first and the second pharmaceutical dosage forms and having the indicia in the dispenser.

In another embodiment, a therapeutic regimen is provided. The therapeutic regimen comprises a first pharmaceutical dosage form comprising a therapeutically effective amount of a first antihistamine and a therapeutically effective amount of an anticholinergic, and a second pharmaceutical dosage form comprising a therapeutically effective amount of a second antihistamine and a therapeutically effective amount of the anticholinergic. The second antihistamine has a greater sedative effect than the first antihistamine. The therapeutic regimen comprises indicia for distinguishing between the first and second pharmaceutical dosage forms, where the indicia distinguishes the first pharmaceutical dosage form for daytime administration and the second pharmaceutical dosage form for nighttime administration. A prepackaged dispenser prefilled with the pharmaceutical dosage forms and comprising the indicia is also provided.

In another embodiment, a method for treating the symptoms of a rhinitic conditions is provided. The method comprises the steps of: formulating a rhinitic treatment regimen comprising a first pharmaceutical dosage form comprising a therapeutically effective amount of a first antihistamine and a therapeutically effective amount of an anticholinergic, and a second pharmaceutical dosage form comprising a therapeutically effective amount of a second antihistamine and a therapeutically effective amount of the anticholinergic. The second antihistamine has a greater sedative effect than the first antihistamine. The method provides indicia for distinguishing between the first and second pharmaceutical dosage forms, where the indicia distinguishes the first pharmaceutical dosage form for daytime administration and the second pharmaceutical dosage form for nighttime administration and prefilling a pharmaceutical dispenser with the first and the second pharmaceutical dosage forms and having the indicia in the dispenser.

### BRIEF DESCRIPTION OF THE DRAWING

For a fuller understanding of the nature and object of the present invention, reference is made to the accompanying drawing, wherein:

FIG. 1 is a perspective view of the front side of an exemplary embodiment of a pharmaceutical dosage form dispenser.

### DETAILED DESCRIPTION

The present invention provides for a prefilled, pharmaceutical dosage form dispenser containing at least two compositionally different pharmaceutical dosage forms for the treatment of rhinitis and indicia for distinguishing the pharmaceutical dosage forms and signifying their use together. Either single or multiple dispensers of each pharmaceutical dosage form may be contained as is desired. The pharmaceutical dosage forms may be in the form of tablets, capsules, lozenges or gelcaps, some of which may require reconstituting, or any generally recognized oral form of medication.

As used herein, “pharmacologically active agent” or “active agent” are used interchangeably and refer to a compound or composition of matter that when administered to a subject (human or animal) causes a desired pharmacologic and/or physiologic effect by local and/or systemic action.

As used herein, “pharmaceutical dosage form” refers to a dosage form of an active agent (e.g., sublingual, chewable, buccal and extended delivery tablets (EDT) forms, tablets, capsules, lozenges, gelcaps, powders and oral care strips/film) which is generally safe, non-toxic and not biologically or otherwise undesirable. A pharmaceutical dosage form includes that which is acceptable for veterinary and/or human pharmaceutical use, and which possesses the necessary and desirable characteristics of a dosage form acceptable for administration to a subject (e.g., a tablet of acceptable hardness, dissolution, stability, and a size and weight practical for oral administration).

As used herein, the term “antihistamine” refers to a moiety that inhibits reduces effects mediated by histamine. By way of example, moieties having negative modulation of histamine receptors as their main therapeutic effect are antihistamines. For example, chlorpheniramine, which may also have anticholinergic activity, is considered an antihistamine. Antihistamine includes, for example, first generation antihistamines such as ethylenediamines, ethanolamines, alkylamines, piperazines and tricyclics. Antihistamines further include acrivastine, astemizole, cetirizine, loratadine, mizolastine, desloratidine and fexofenadine.

As used herein, the term “H₁ receptor activity” refers to a level of inhibition of binding to or interacting with a H₁ receptor. The term H₁ receptor activity includes H₁ receptor antagonistic activity and H₁ receptor inverse agonistic activity. Determining H₁ receptor activity is known, see, e.g., V. T. Tran et al, “Histamine H₁ receptors identified in mammalian brain membranes with [³H]mepyramine”, Proc. Natl. Acad. Sci. (1978) 75: 6290-94. The Tran et al. document is incorporated by reference herein in its entirety.

The term “wherein the second antihistamine H₁ receptor activity is greater than the first antihistamine H₁ receptor activity” includes a regimen where the first and second antihistamines are different antihistamines or includes a regimen where the first and second antihistamines are the same antihistamine, but in a larger dose in the case of the second antihistamine. For example, two different antihistamines may be used provided that the second antihistamine has greater H₁ receptor activity than the first. Also, the same antihistamine may be used for both first and second antihistamines provided that the amount of the second is greater than the first.

As used herein, the term “anticholinergic” refers to a moiety that reduces or inhibits effects of acetylcholine on the central nervous system or peripheral nervous system. For example, moieties having as their main therapeutic effect reversible inhibition of muscarinic acetylcholine receptors or nicotinic acetylcholine receptors are considered anticholinergics. Anticholinergics with limited capacity to cross the blood-brain barrier are preferred, for example, methscopolamine salts and glycopyrrolate.

As used herein, “pharmacological effect” encompasses pharmacokinetic and pharmacodynamic effects produced in a subject that achieve the intended purpose of a therapy or regimen. By way of example, a pharmacological effect means that rhinitis symptoms of the subject being treated are prevented, alleviated, or reduced. In one example, a pharmacological effect would include inhibition of binding or interacting with a H₁ receptor. In another example, a pharmacological effect would include one that results in the prevention or reduction of hypertension in a treated subject.

As used herein, the terms “daytime” and “nightime” are intended to include literally day and night, in that such times vary in accordance with the schedule of the individual.

As used herein, the term “rhinitic condition” refers to the symptomology of rhinitis. Symptomology of rhinitis includes, for example, repetitive sneezing, rhinorrhea (runny nose), post-nasal drip, nasal congestion, pruritic (itchy) eyes, ears, nose or throat, and generalized fatigue. Rhinitic condition may also include wheezing, eye tearing, sore throat, impaired smell, sinus headaches and ear plugging.

Subjects suffering from rhinitic conditions generally display pale or violaceous nasal mucosa because of the engorged veins, nasal polyps, swelling of the eyelids, injected sclerae (the whites of the eyes may be red), allergic shiners (darkened areas under the lower eyelids thought to result from venous pooling of blood), and extra skin folds in the lower eyelids.

Factors which may cause rhinitis include year-round allergens, for example, dust mites, cockroaches, molds and animal dander, and seasonal outdoor allergens, for example, tree, grass and ragweed pollens. Indoor allergens include, for example, mold spores and animal allergens.

As used herein, “sympathomimetic decongestant” refers generally to a class of decongestant whose pharmacology includes mimicking a stimulated sympathetic central nervous system. The effect of sympathomimetic decongestant includes, for example, increased cardiac output, dilation of bronchioles, and constriction of blood vessels. Sympathomimetic decongestants include, for example phenylephrine, pseudoephedrine and phenylpropanolamine.

As used herein, “sedative effect” refers to a soporific property of an antihistamine. Determining a soporific property of an antihistamines is known, see e.g., Witek, et al., “Characterization of daytime sleepiness and psychomotor performance following H₁ receptor antagonists,” Annals of Allergy, Asthma, & Immunology (1995) 74: 419-26, the disclosure of which is incorporated by reference in its entirety.

The formulation of therapeutically effective combinations requires pharmaceutical expertise and understanding of the pharmacokinetic and pharmacodynamic actions and side effects of antihistamines, anticholinergics, and other formulated components, including components which may affect the pharmacokinetic and pharmacodynamic bioavailability and effectiveness of the active ingredients, and their suitability of use together. It is therefore another embodiment to provide a subject with an effective regimen that maximizes the pharmacokinetic and pharmacodynamic bioavailability and effectiveness of the regimen.

It is another embodiment to provide a method and device for organizing, storing, and coordinating regimens for the treatment of rhinitis for the purpose of convenience in using such regimens by providing such regimens, prepackaged in such a way that incorporates coordinating indicia and administration of the pharmaceutical dosage forms.

It is another embodiment to provide regimens which provide a therapeutic combination of anticholinergic and an antihistamine dosage during the night in accordance with its pharmacokinetic and pharmacodynamic profile so as to achieve histamine receptor binding in the morning hours, at the time of awakening.

In one embodiment, a user is provided with a therapeutic combination of anticholinergic and attenuated first antihistamine H₁ receptor activity during the day, which is suitable with respect to its half-life, duration of action, and duration of side effect, and a dosing of a second antihistamine having greater H₁ receptor activity than the first antihistamine with the anticholinergic at nighttime.

In one embodiment, a prefilled, pharmaceutical dosage form dispenser is provided containing at least two compositionally different pharmaceutical dosage forms for the treatment of rhinitis, and indicia for distinguishing the pharmaceutical dosage forms. The dispenser can have one of any number of forms, including, but not limited to, a box with the dosage forms in bottles, a blister package, a box of individual blister packages, or a box of pouches.

The embodiments of the dispenser will become apparent in light of the following drawing and detailed description thereof.

Referring to the drawing, it will be understood that while various embodiments of the invention have been illustrated and described, the invention is not limited to such embodiments. Changes and additions may be made therein and thereto without departing from the spirit of the invention.

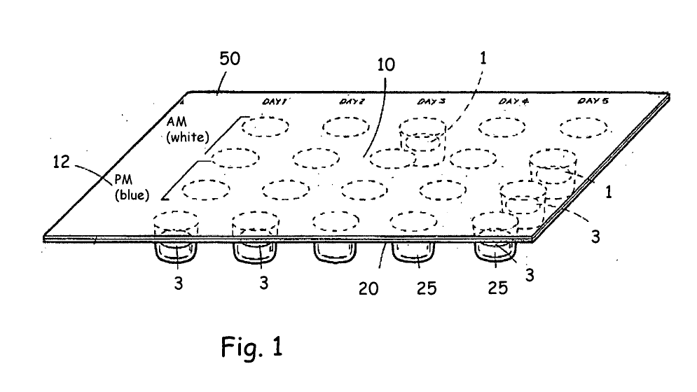

Referring to FIG. 1, the container is in the form of a blister pack dispenser comprising a frontside surface 10 with indicia 12 defined thereon; a blister film 20 affixed to the frontside surface 10 having held therein two types of discrete pharmaceutical dosage forms 1 and 3.

The blister pack dispenser shown in FIG. 1 is an elongate rectangle. The blister pack dispenser may be of any acceptable shape. The indicia 12 is defined on the frontside surface 10 by the arrangement of printed words or other symbols along two axes, the first axis being alongside one edge of the frontside surface 10, the other axis being at right angles to the first. The first axis is defined by the words “Day 1”, “Day 2”, etc., and may be regularly spaced, these words representing, for example, consecutive days or administration periods of the therapy or regimen. The other axis may be defined by the words “AM” and “PM”, or other well recognized equivalent symbols representing daytime and night-time within each day/period of the therapy or regimen.

The front side surface 10 has an area 50, outside the indicia 12, on which may be printed administration instructions. Administration instructions may include words or well recognized symbols that convey to the user the intended administration therapy or regimen. Alternatively, the section may be intentionally left blank, for example, to allow a health care provider to write specific instructions.

The blister film generally indicated at 20 may be of a conventional blister pack dispenser type, in which blisters 25 may be formed in a regular grid of rows and columns.

The grid of blisters 25 may be so arranged in the blister film 20 that a column of blisters 25 is in register with each of “Day 1”, “Day 2”, etc. on the front side surface 10 and rows of blisters are in register with “AM” and with “PM” on the front side surface 10.

The two types of discrete pharmaceutical dosage forms generally indicated at 1 and 3 may be located in the closed blisters 25 of the blister film 20. Pharmaceutical dosage forms 1 may comprise a composition comprising an anticholinergic and an attenuated dosage of the first antihistamine and packed in the two rows of blisters 25 in register with the word “AM” in the indicia 12. Pharmaceutical dosage forms of the pharmaceutical dosage form 3 that include the second antihistamine, which may be sedative, and the anticholinergic are packed in the rank of blisters 25 in register with the word “PM” in the indicia 12. The pharmaceutical dosage forms of the pharmaceutical dosage form 1 may be of a different color to those of pharmaceutical dosage form 3.

The order of packing of the pharmaceutical dosage forms of types 1 and 3 located in the blisters 25 of the blister film 20 in register with the indicia 12, and the different colors of the two dosage types facilitate the taking of pharmaceutical dosage forms 1 by day and pharmaceutical dosage form 3 at night.

In addition to indicating and facilitating the taking of the various pharmaceutical dosage forms in accordance with a desired treatment regime, the dispenser illustrated also conveniently shows when the necessary pharmaceutical dosage forms have been taken.

To remove any pharmaceutical dosage form 1 and 3 at a time indicated as appropriate as above the corresponding blister 25 containing it may be accomplished by pressing with a finger to push the pharmaceutical dosage form through the front side surface 10.

The base and blister film of the blister pack dispenser may be of any materials suitable for the construction of blister packs, for example, an aluminum foil base and a thermoplastic blister film.

Although the administration instructions may be printed on the base, they may also be written or printed on a separate surface such as a sheet of paper, or on a label attached to the pack.

Although the illustrated dispenser specifically described is generally for up to a five-day dosage regime, it is envisaged that the dispenser may be adapted for longer or shorter periods of time, as desired, merely by shortening or lengthening the dispenser and correspondingly decreasing or increasing the number of columns of blisters as appropriate.

Further, although the illustrated dispenser specifically described is for a regime of a pharmaceutical dosage forms for day-time use and for night-time use, it is envisaged that the desired regime may specify any number of pharmaceutical dosage forms for each aspect of the therapy. Consequently, the dispenser may be adapted in accordance with the requirements of the regime by narrowing or widening the dispenser and correspondingly decreasing or increasing the number of rows of blisters and the number of rows in register with “AM” and “PM” as appropriate.

The blister pack dispenser described has an indicia defined on it in the form of rows and columns with corresponding positioning of the pharmaceutical dosage form containing blisters. The indicia, and corresponding blisters, may be in any geometric configuration provided that the indicia clearly indicates which pharmaceutical dosage forms are to be taken during the day and which pharmaceutical dosage forms are to be taken at night. Also, the indicia may be omitted, but in this case pharmaceutical dosage forms of the different types must have a visible distinguishing feature, such as a difference in color, to indicate that they relate to different aspects of the dosage regime. The indicia and such a distinguishing feature may both be present. One or more blister packs may be housed in any suitable form for dispensing.

The dispenser embodiments described herein are not limited to blister packs. Thus, any conventional pharmaceutical containers are suitable. Examples thereof include bottles, tubes, canisters and packets. Where such containers do not readily permit the housing of the pharmaceutical dosage forms in register with a indicia, for example bottles, the pharmaceutical dosage forms must be mutually distinguished by some visible feature, such as a difference in color, form, shape or size, or by marks or printing therein, to indicate which pharmaceutical dosage forms are for day-time and which pharmaceutical dosage forms are for night-time.

The packaging may contain combinations of medications, which include anticholinergics and antihistamines, which comprise a regimen for treating rhinitis. For example, the packaged medication may be comprised of at least two pharmaceutical dosage forms, one for administration when sedation is not desired, as, for example, during the day, and one for administration in anticipation of peak histamine release and/or when sedation is desired, as, for example, at night. The regimen is devised utilizing a combination of pharmaceutical dosage forms which are favorable for use with each other, particularly with regard to pharmacokinetic and therapeutic characteristics. It is possible to formulate these regimens with presently available pharmaceutical dosage forms or active agents as well as with newly formulated pharmaceutical dosage forms or active agents. Examples of regimens, some of which employ presently available pharmaceutical dosage forms and active agents, are described below.

There are a number of possible combinations of anticholinergics and antihistamines presently available that can be employed in the present invention. Other medications, such as analgesics and non-sympathomimics may be incorporated into the dosage forms.

The embodiments of the regimen will become apparent in light of the following examples. The following examples are illustrative and not to be interpreted as limiting or restrictive. Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements.

### EXAMPLE 1

A first pharmaceutical dosage form comprising about 2.5 milligrams methscopolamine nitrate and about 4 milligrams of chlorpheniramine maleate may be provided in tablet form to be taken in the morning. This tablet may be colored white, for example. A second pharmaceutical dosage form of about 2.5 milligrams methscopolamine nitrate and about 8 milligrams of chlorpheniramine maleate may be provided in tablet form to be taken at bedtime. This tablet may be colored blue. The amount of chlorpheniramine maleate in the second tablet therefore would provide greater H₁ receptor activity.

### EXAMPLE 2

A first pharmaceutical dosage form comprising about 2.5 milligrams methscopolamine nitrate (anticholinergic) and about 6 milligrams brompheniramine maleate (antihistamine) may be provided in tablet form to be taken in the morning. This tablet may be colored white, for example. A second pharmaceutical dosage form of about 2.5 milligrams methscopolamine nitrate and about 25 milligrams diphenhydramine hydrochloride may be provided in tablet form to be taken at bedtime. This tablet may be colored blue, for example. The amount of diphenhydramine hydrochloride in the second tablet would provide for greater H₁ receptor activity than the amount of brompheniramine maleate in the first tablet.

### EXAMPLE 3

A first pharmaceutical dosage form comprising about 2.5 milligrams methscopolamine nitrate and about 3 milligrams dexchlorpheniramine maleate (antihistamine) may be provided in tablet form to be taken in the morning. This tablet may be colored white, for example. A second pharmaceutical dosage form of about 2.5 milligrams methscopolamine nitrate and about 6 milligrams dexchlorpheniramine maleate may be provided in tablet form to be taken at bedtime. This tablet may be colored blue, for example. The amount of dexchlorpheniramine maleate in the second tablet therefore would provide greater H₁ receptor activity.

### EXAMPLE 4

A first pharmaceutical dosage form comprising about 1.25 milligrams methscopolamine nitrate and about 2 milligrams chlorpheniramine maleate (antihistamine) may be provided in tablet form to be taken in the morning. This tablet may be colored white, for example. A second pharmaceutical dosage form of about 1.25 milligrams methscopolamine nitrate and about 4 milligrams chlorpheniramine maleate hydrochloride may be provided in tablet form to be taken at bedtime. This tablet may be colored blue, for example. The amount of chlorpheniramine maleate in the second tablet therefore would provide greater H₁ receptor activity.

### EXAMPLE 5

A first pharmaceutical dosage form comprising about 2.5 milligrams methscopolamine nitrate and about 5 milligrams desloratidine (antihistamine) may be provided in tablet form to be taken in the morning. This tablet may be colored white, for example. A second pharmaceutical dosage form of about 2.5 milligrams methscopolamine nitrate and about 8 milligrams chlorpheniramine maleate may be provided in tablet form to be taken at bedtime. This tablet may be colored blue, for example. The antihistamine in the second pharmaceutical dosage form would have a greater sedative effect than the first pharmaceutical dosage form.

### EXAMPLE 6

A first pharmaceutical dosage form comprising about 2.5 milligrams methscopolamine nitrate and about 180 milligrams fexofenadine (antihistamine) may be provided in tablet form to be taken in the morning. This tablet may be colored white, for example. A second pharmaceutical dosage form of about 2.5 milligrams methscopolamine nitrate and about 25 milligrams diphenhydramine hydrochloride may be provided in tablet form to be taken at bedtime. This tablet may be colored blue, for example. The antihistamine in the second pharmaceutical dosage form would have a greater sedative effect than the first pharmaceutical dosage form.

Despite an indication for 4 to 6 hour dosing for chlorpheniramine, a single dose may inhibit the symptoms of rhinitis for more than 24 hours. Maximal pharmacological sedation peaks approximately 3 to 4 hour after dosing and sedation persists usually not longer than 6 to 8 hours following dosing.

The combination of an attenuated antihistamine dosing and anticholinergic during the day and greater pharmacologic antihistamine dosing with anticholinergic dosing at night pharmacologically maximizes the short duration of sedation in relation to the longer duration of rhinitic symptom suppression while avoiding the risk of hypertension (EXAMPLES 1-4). This combined dosage regimen anticipates peak morning histamine release and effectively confers combined antihistamine and anticholinergic activity during the day with minimal sedation as a result of pharmacologically adjusted dosing of the first antihistamine during the day.

Alternatively, the combination of non-sedative antihistamine dosing and anticholinergic during the day and sedative antihistamine dosing with anticholinergic dosing at night reduces or eliminates daytime sedation in relation to the longer duration of rhinitic symptom suppression while avoiding the risk of hypertension (EXAMPLES 5-6). The absence of a sympathomimetic decongestant dosing avoids the potential for hypertension stimulation and insomnia at night. This combined dosage regimen anticipates peak morning histamine release and effectively confers combined antihistamine and anticholinergic activity during the day with minimal sedation as a result of pharmacologically reducing or eliminating sedation with the first antihistamine dosage during the day while pharmacologically providing a longer duration of rhinitic symptom suppression with the second antihistamine dosage.

In addition to antihistamines and anticholinergic active agents, other therapeutic ingredients for the treatment of rhinitis may be formulated if desired. For example, analgesics such as salicylates and acetominifen may be included. In addition to the pharmacologically active agents, pharmaceutical dosage forms may contain a number of inert materials or additives. Inert materials and additives may include materials that help in the manufacture of the tablet or to impart satisfactory compression characteristics to the formulation. Inert materials and additives may also include materials that help to give additional desirable physical characteristics to the dosage form, such as colors, flavors, and sweetening agents. Such inert materials and additives should not materially affect the pharmacological properties of the active agent or agents.

Pharmaceutical dosage forms may contain one or more excipients or vehicles chosen from diluents, lubricants, binders, disintegrating agents, absorbents, and the like. The dissolution rate of a pharmaceutical dosage form may be increased by the addition of disintegrant or solubilizing substances.

As used herein, “comprising,” “including,” “containing,” “characterized by,” and grammatical equivalents thereof are inclusive or open-ended terms that do not exclude additional, unrecited elements or method steps. “Comprising” is to be interpreted as including the more restrictive terms “consisting of” and “consisting essentially of.”

As used herein, “consisting of” and grammatical equivalents thereof exclude any element, step, or ingredient not specified in the claim.

As used herein, “consisting essentially of” and grammatical equivalents thereof limit the scope of a claim to the specified materials or steps and those that do not materially affect the basic and novel characteristic or characteristics of the claimed invention.

Other variations may occur to those skilled in the art which are within the scope of the invention as set forth in the appended claims. Those of skill in the art may also recognize modifications to these presently disclosed embodiments. These variations and modifications are meant to be covered by the spirit and scope of the present claims.

We claim:

1. A therapeutic regimen comprising:
a first pharmaceutical dosage form comprising a first therapeutically effective amount of a first antihistamine having H₁ receptor activity and a therapeutically effective amount of an anticholinergic;
a second pharmaceutical dosage form comprising a second therapeutically effective amount of a second antihistamine having H₁ receptor activity and a therapeutically effective amount of the anticholinergic, wherein the second antihistamine H₁ receptor activity is greater than the first antihistamine H₁ receptor activity, with the proviso that the second antihistamine is not acrivastine, astemizole, cetirizine, loratadine, mizolastine, desloratidine or fexofenadine;
indicia for distinguishing between the first and second pharmaceutical dosage forms; wherein the indicia distinguishes the first pharmaceutical dosage form for daytime administration and the second pharmaceutical dosage form for nighttime administration; and
a prepackaged dispenser prefilled with the pharmaceutical dosage forms and comprising the indicia.

2. The therapeutic regimen of claim 1, wherein the first and second pharmaceutical dosage forms are essentially absent of a sympathomimetic decongestant.

3. The therapeutic regimen of claim 1, wherein the second antihistamine is sedating.

4. The therapeutic regimen of claim 1, wherein the first or second antihistamine is chlorpheniramine maleate, brompheniramine maleate, diphenhydramine hydrochloride or dexchlorpheniramine maleate.

5. The therapeutic regimen of claim 3, wherein:
the first antihistamine amount is about 4 milligrams and the second antihistamine amount is about 8 milligrams when the antihistamine is chlorpheniramine maleate;
the first antihistamine amount is about 3 milligrams and the second antihistamine amount is about 6 milligrams when the antihistamine is dexchlorpheniramine maleate; and
the first antihistamine amount is about 6 milligrams when the first antihistamine is brompheniramine maleate and the second antihistamine amount is about 25 milligrams when the second antihistamine is diphenhydramine hydrochloride.

6. The therapeutic regimen of claim 1, wherein the anticholinergic is methscolopamine nitrate.

7. The therapeutic regimen of claim 5, wherein the amount of methscolopamine nitrate is between about 1.25 milligrams to about 2.5 milligrams.

8. The therapeutic regimen of claim 1, wherein the dispensing package is a blister pack.

9. The therapeutic regimen of claim 1, wherein the pharmaceutical dosage forms comprises tablets, capsules, lozenges or gelcaps.

10. The therapeutic regimen of claim 1, wherein the first pharmaceutical dosage form consists essentially of the first antihistamine and the anticholinergic and the second pharmaceutical dosage form consists essentially of the second antihistamine and the anticholinergic.

11. The therapeutic regimen of claim 10, wherein the pharmaceutical dosage forms are tablets, capsules, lozenges or gelcaps.

12. A method for treating the symptoms of a rhinitic conditions comprising the steps of:
formulating a rhinitic treatment regimen comprising a first pharmaceutical dosage form comprising a therapeutically effective amount of a first antihistamine having H₁ receptor activity and a therapeutically effective amount of an anticholinergic, and a second pharmaceutical dosage form comprising a therapeutically effective amount of a second antihistamine having H₁ receptor activity and a therapeutically effective amount of the anticholinergic; wherein the second antihistamine H₁ receptor activity greater than the first antihistamine H₁ receptor activity;
providing indicia for distinguishing between the first and second pharmaceutical dosage forms; wherein the indicia distinguishes the first pharmaceutical dosage form for daytime administration and the second pharmaceutical dosage form for nighttime administration; and
prefilling a pharmaceutical dispenser with the first and the second pharmaceutical dosage forms and having the indicia in the dispenser.

13. The method of claim 12, wherein the first and second pharmaceutical dosage forms are essentially absent of a sympathomimetic decongestant.

14. The method of claim 12, wherein the second antihistamine is sedating.

15. The method of claim 12, wherein the first or second antihistamine is chlorpheniramine maleate, brompheniramine maleate, diphenhydramine hydrochloride or dexchlorpheniramine maleate.

16. The method of claim 12, wherein:
the first antihistamine amount is about 4 milligrams and the second antihistamine amount is about 8 milligrams when the antihistamine is chlorpheniramine maleate;
the first antihistamine amount is about 3 milligrams and the second antihistamine amount is about 6 milligrams when the antihistamine is dexchlorpheniramine maleate; and
the first antihistamine amount is about 6 milligrams when the first antihistamine is brompheniramine maleate and the second antihistamine amount is about 25 milligrams when the second antihistamine is diphenhydramine hydrochloride.

17. The method of claim 12, wherein the anticholinergic is methscolopamine nitrate.

18. The method of claim 17, wherein the amount of methscolopamine nitrate is between about 1.25 milligrams to about 2.5 milligrams.

19. The method of claim 12, wherein the dispensing package is a blister pack.

20. The method of claim 12, wherein the pharmaceutical dosage forms comprises tablets, capsules, lozenges or gelcaps

21. The method of claim 12, wherein the first pharmaceutical dosage form consists essentially of the first antihistamine and the anticholinergic and the second pharmaceutical dosage form consists essentially of the second antihistamine and the anticholinergic.

22. The method of claim 12, wherein the regimen reduces or eliminates hypertension in a subject in comparison with a regimen comprising an antihistamine and a sympathomimetic decongestant.

23. A therapeutic regimen comprising:
a first pharmaceutical dosage form comprising a therapeutically effective amount of a first antihistamine and a therapeutically effective amount of an anticholinergic;
a second pharmaceutical dosage form comprising a therapeutically effective amount of a second antihistamine and a therapeutically effective amount of the anticholinergic, wherein the second antihistamine has a greater sedative effect than the first antihistamine;
indicia for distinguishing between the first and second pharmaceutical dosage forms; wherein the indicia distinguishes the first pharmaceutical dosage form for daytime administration and the second pharmaceutical dosage form for nighttime administration; and
a prepackaged dispenser prefilled with the pharmaceutical dosage forms and comprising the indicia.

24. The therapeutic regimen of claim 23, wherein the first antihistamine is selected from acrivastine, astemizole, cetirizine, loratadine, mizolastine, desloratidine and fexofenadine, and the second antihistamine is selected from chlorpheniramine maleate, brompheniramine maleate, diphenhydramine hydrochloride and dexchlorpheniramine maleate.

25. A method for treating the symptoms of a rhinitic conditions comprising the steps of:
formulating a rhinitic treatment regimen comprising a first pharmaceutical dosage form comprising a therapeutically effective amount of a first antihistamine and a therapeutically effective amount of an anticholinergic, and a second pharmaceutical dosage form comprising a therapeutically effective amount of a second antihistamine and a therapeutically effective amount of the anticholinergic; wherein the second antihistamine has a greater sedative effect than the first antihistamine;
providing indicia for distinguishing between the first and second pharmaceutical dosage forms; wherein the indicia distinguishes the first pharmaceutical dosage form for daytime administration and the second pharmaceutical dosage form for nighttime administration; and
prefilling a pharmaceutical dispenser with the first and the second pharmaceutical dosage forms and having the indicia in the dispenser.

26. The method of claim 25, wherein the first antihistamine is selected from acrivastine, astemizole, cetirizine, loratadine, mizolastine, desloratidine and fexofenadine, and the second antihistamine is selected from chlorpheniramine maleate, brompheniramine maleate, diphenhydramine hydrochloride and dexchlorpheniramine maleate.

27. The method of claim 25, wherein the regimen reduces or eliminates hypertension in a subject in comparison with a regimen comprising an antihistamine and a sympathomimetic decongestant.
